(19) United States
(12) Patent Application Publication
(10) Pub. No.: US 2024/0201792 A1
(43) Pub. Date: Jun. 20, 2024

(54) FULL HAND KINEMATICS RECONSTRUCTION USING ELECTRICAL IMPEDANCE TOMOGRAPHY WEARABLE

(71) Applicant: GOOGLE LLC, Mountain View, CA (US)

(72) Inventors: Stiven Guillaume Francois Morvan, New York, NY (US); Dongeek Shin, San Jose, CA (US); Andrea Colaco, Los Altos, CA (US); Sambuddha Basu, San Jose, CA (US); Sean Kyungmok Bae, San Francisco, CA (US); Junyi Zhu, Cambridge, MA (US)

(21) Appl. No.: 18/537,224

(22) Filed: Dec. 12, 2023

Related U.S. Application Data
(60) Provisional application No. 63/387,443, filed on Dec. 14, 2022.

(51) Int. Cl. G06F 3/01; A61B 5/0536; A61B 5/263; G06V 10/12; G06V 10/764; G06V 40/20

(52) U.S. Cl. CPC G06F 3/017; A61B 5/0536; A61B 5/263; G06V 10/12; G06V 10/764; G06V 40/28

(57) ABSTRACT

Techniques include determining hand gestures formed by a user based on an electrical impedance tomograph of the wrist. For example, a user may be outfitted with a flexible wristband that fits snugly around the wrist and contains a plurality of electrodes, e.g., 32 electrodes. When a current is applied to a first subset of the electrodes, e.g., two of 32 electrodes, the electric field induced through at least one cross-section of the wrist will in turn induce a voltage across adjacent pairs of a second subset of the electrodes (e.g., the other 30 of 32 electrodes). From this current and induced voltage, one may use techniques of electrical impedance tomography (EIT) to determine the electrical impedance throughout the at least one cross-section of the wrist, e.g., in an electrical impedance tomograph. One may use a neural network to map the electrical impedance tomograph to a hand gesture.

### CROSS REFERENCE TO RELATED APPLICATION

This application claims the benefit of U.S. Provisional Application No. 63/387,443, filed Dec. 14, 2022, the disclosure of which is incorporated herein by reference in its entirety.

### TECHNICAL FIELD

This description relates in general to head mounted wearable devices, and in particular, to determination of continuous finger positions for controlling head mounted wearable computing devices including a display device.

### SUMMARY

The improvement discussed herein is directed to determining continuous hand kinematics in 3D space formed by a user based on an electrical impedance tomograph of the wrist. For example, a user may be outfitted with a flexible wristband that fits snugly around the wrist and contains a plurality of electrodes, e.g., 32 electrodes. When a current is applied to a first subset of the electrodes, e.g., two of 32 electrodes, the electric field induced through at least one cross-section of the wrist will in turn induce a voltage across adjacent pairs of a second subset of the electrodes (e.g., the other 30 of 32 electrodes). This process can be repeated multiple times until all subsets of the electrodes, e.g., all 32 pairs of electrodes, have been applied current, with induced voltage measured across remaining non-current injecting electrodes successively in each round. From these currents and induced voltages, one may use techniques of electrical impedance tomography (EIT) to determine the electrical impedance throughout at least one cross-section of the wrist, e.g., in an electrical impedance tomograph. One may use a machine learning engine, e.g., a neural network, to map the electrical impedance tomograph to a five finger positions in 3D space.

In one general aspect, a method includes receiving an electrical impedance tomograph representing a map of electrical impedance through at least one cross-section of a wrist of a user; determining a gesture formed by a hand of the user based on the electrical impedance tomograph; and triggering execution of a command related to an object being displayed in an augmented reality (AR) system based on the gesture.

In another general aspect, an AR system includes gesture detection circuitry coupled to a memory, the gesture detection circuitry being configured to receive an electrical impedance tomograph representing a map of electrical impedance through at least one cross-section of a wrist of a user; and determine a gesture formed by a hand of the user based on the electrical impedance tomograph, wherein the AR system is configured to trigger execution of a command related to an object being displayed in the AR system based on the gesture.

In another general aspect, a computer program product comprising a nontransitory storage medium, the computer program product including code that, when executed by processing circuitry, causes the processing circuitry to perform a method, the method including receiving an electrical impedance tomograph representing a map of electrical impedance through at least one cross-section of a wrist of a user; determining a gesture formed by a hand of the user based on the electrical impedance tomograph; and triggering execution of a command related to an object being displayed in an augmented reality (AR) system based on the gesture.

The details of one or more implementations are set forth in the accompanying drawings and the description below. Other features will be apparent from the description and drawings, and from the claims.

### BRIEF DESCRIPTION OF THE DRAWINGS

FIG. 1A illustrates an example system, in accordance with implementations described herein.

FIG. 1B is a front view, FIG. 1C is a rear view, and FIG. 1D is a perspective view, of the example head mounted wearable device shown in FIG. 1A, in accordance with implementations described herein.

FIG. 2 is a diagram illustrating an example generation of an electrical impedance tomograph.

FIG. 3 is a diagram illustrating an example snug wristband with electrodes for determining an electrical impedance tomograph.

FIGS. 4A and 4B are diagrams illustrating example gesture classification using keypoints in a human hand.

FIG. 5 is a diagram illustrating an example electronic environment for performing determination of a gesture based on an electrical impedance tomograph.

FIG. 6 is a flow chart illustrating a method of determining a gesture based on an electrical impedance tomograph.

### DETAILED DESCRIPTION

A challenge for AR systems is providing a robust interface between the smartglasses and the user. Some interfaces utilize user gestures to effect various commands. A robust interface may be able to distinguish between a thumb-index pinch and a thumb-middle finger pinch.

A conventional approach to providing a robust AR interface includes using a world-facing RGB camera mounted on a smartglasses frame to provide images from which a skeletal hand track for full hand/finger interaction and gesture deduction. Nevertheless, there can be issues related to the use of a world-facing RGB camera. For example, such a world-facing RGB camera mounted on a frame of the smartglasses, usually near a hinge, has a camera sensor and an image stack processor (ISP) that consume a relatively large amount of power. Due to this large power consumption, a technical problem with using RBG images from the world-facing RGB camera to deduce user gestures is that the world-facing RGB camera can only be used sparingly.

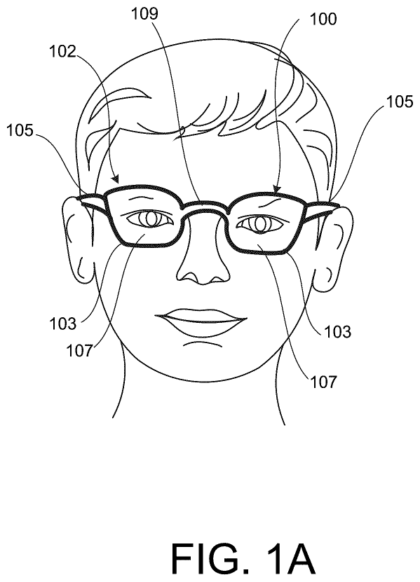

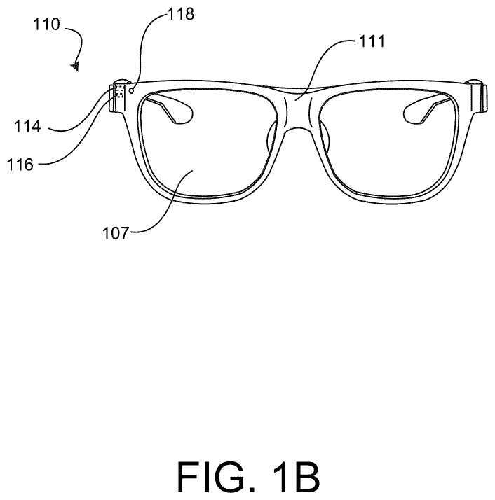

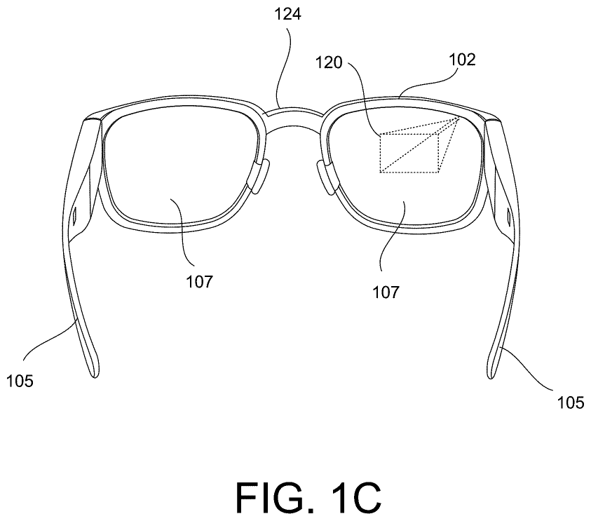

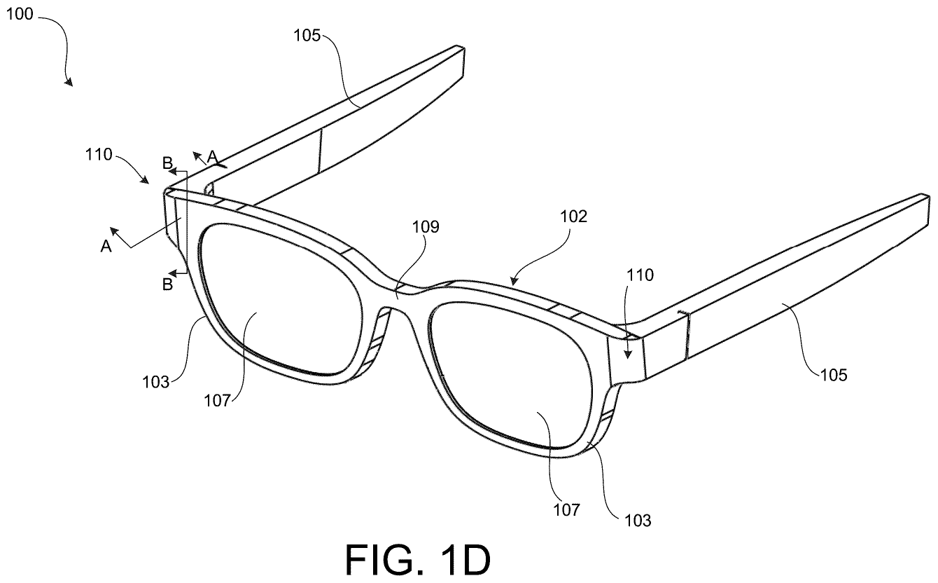

A technical solution to the above-described technical problem includes determining hand gestures formed by a user based on an electrical impedance tomograph of the wrist. An example AR system that can be used in connection with the technical solution are described in FIGS. 1A-1D.

FIG. 1A illustrates a user wearing an example smartglasses 100. In this example, the example smartglasses 100 including display capability and computing/processing capability, for purposes of discussion and illustration. The principles to be described herein may be applied to other types of eyewear, both with and without display capability and/or computing/processing capability. FIG. 1B is a front view, FIG. 1C is a rear view, and FIG. 1D is a perspective view, of the smartglasses 100 shown in FIG. 1A. The above-described gestures determined from electrical impedance tomographs in the form of continuous hand kinematics may be used to trigger execution of commands related to objects being displayed in smartglasses 100.

As shown in FIG. 1B-1D, the example smartglasses 100 includes a frame 102. The frame 102 includes a front frame portion defined by rim portions 103 surrounding respective optical portions in the form of lenses 107, with a bridge portion 109 connecting the rim portions 109. Arm portions 105 are coupled, for example, pivotably or rotatably coupled, to the front frame by hinge portions 110 at the respective rim portion 103. In some examples, the lenses 107 may be corrective/prescription lenses. In some examples, the lenses 107 may be an optical material including glass and/or plastic portions that do not necessarily incorporate corrective/prescription parameters. A display device 104 may be coupled in a portion of the frame 102. In the example shown in FIGS. 1B and 1C, the display device 104 is coupled in the arm portion 105 of the frame 102. In some examples, the smartglasses 100 can also include an audio output device 106 (such as, for example, one or more speakers), an illumination device 108, a sensing system 111, a control system 112, at least one processor 114, and an outward facing image sensor, or camera 116. In some examples, the display device 104 may include a see-through near-eye display. For example, the display device 104 may be configured to project light from a display source onto a portion of teleprompter glass functioning as a beamsplitter seated at an angle (e.g., 30-45 degrees). The beamsplitter may allow for reflection and transmission values that allow the light from the display source to be partially reflected while the remaining light is transmitted through. Such an optic design may allow a user to see both physical items in the world, for example, through the lenses 107, next to content (for example, digital images, user interface elements, virtual content, and the like) generated by the display device 104. In some implementations, waveguide optics may be used to depict content on the display device 104 via outcoupled light 120.

The challenge for AR systems as mentioned above is providing a robust interface between the smartglasses 100 and the user. Some interfaces utilize user gestures to effect various commands. A robust interface may be able to distinguish between a thumb-index pinch and a thumb-middle finger pinch. For example, the different gestures may indicate activation of different icons on a smartglasses display or indication of different objects to move within a display field.

A conventional approach to providing a robust AR interface includes using a world-facing RGB camera mounted on a smartglasses frame to provide images from which a skeletal hand track for full hand/finger interaction and gesture deduction. That is, the RGB camera mounted on a frame hinge would track hand and/or finger motions of a user's hand and thereby deduce a gesture and effect a command associated with the gesture, e.g., continuous hand kinematics at an instant of time.

Nevertheless, there can be issues related to the use of a world-facing RGB camera. For example, such a world-facing RGB camera mounted on a frame of the smartglasses, usually near a hinge, has a camera sensor and an image stack processor (ISP) that consume a relatively large amount of power. Due to this large power consumption, a technical problem with using RBG images from the world-facing RGB camera to deduce user gestures is that the world-facing RGB camera can only be used sparingly. For example, using the world-facing RGB camera, the on-board sensors can only detect gestures a few times a day based on the camera's power consumption. Moreover, the frame rate—which should be about 30 frames per second to get the sort of resolution for fine hand movement distinctions, may be less than 5 frames per second in operation with a high latency.

Moreover, even if the technical problem of high power consumption could be overcome, there is another technical problem involving possible occlusion within a narrow field of view; if hands and/or fingers are not visible within the display field, then the gesture detection will not work.

There is some evidence that an electrical impedance tomograph of a user's wrist, e.g., a map of electrical impedance through a cross-section of a user's wrist determined through electrical impedance tomography (EIT), contains useful information about hand and finger movements.

In accordance with the implementations described herein, a technical solution to the above-described technical problem includes determining hand gestures formed by a user based on an electrical impedance tomograph of the wrist. For example, a user may be outfitted with a flexible wristband that fits snugly around the wrist and contains a plurality of electrodes, e.g., 32 electrodes. When a current is applied to a first subset of the electrodes, e.g., two of 32 electrodes, the electric field induced through at least one cross-section of the wrist will in turn induce a voltage across adjacent pairs of a second subset of the electrodes (e.g., the other 30 of 32 electrodes). From this current and induced voltage, one may use techniques of electrical impedance tomography (EIT) to determine the electrical impedance throughout the at least one cross-section of the wrist, e.g., in an electrical impedance tomograph. One may use a machine learning engine, e.g., a neural network, to map the electrical impedance tomograph to a hand gesture. This is done, for example, by defining a set of keypoints of a human hand and training the machine learning engine on the location of the set of keypoints in space associated with an electrical impedance tomograph.

A technical advantage of the technical solution is that, in contrast to the conventional approaches, uses sufficiently low power so that the image capture device is always available and able to provide a high frame rate (e.g., 30 frames per second) and low latency (e.g., about 10 milliseconds). Moreover, occlusion is no longer an issue as the illumination and detector are positioned a few millimeters from the wrist. Specifically, a z-depth of a band on which the image capture device is mounted is controlled by the z-depth of the image capture device than, say, a mount height on the smartglasses frame.

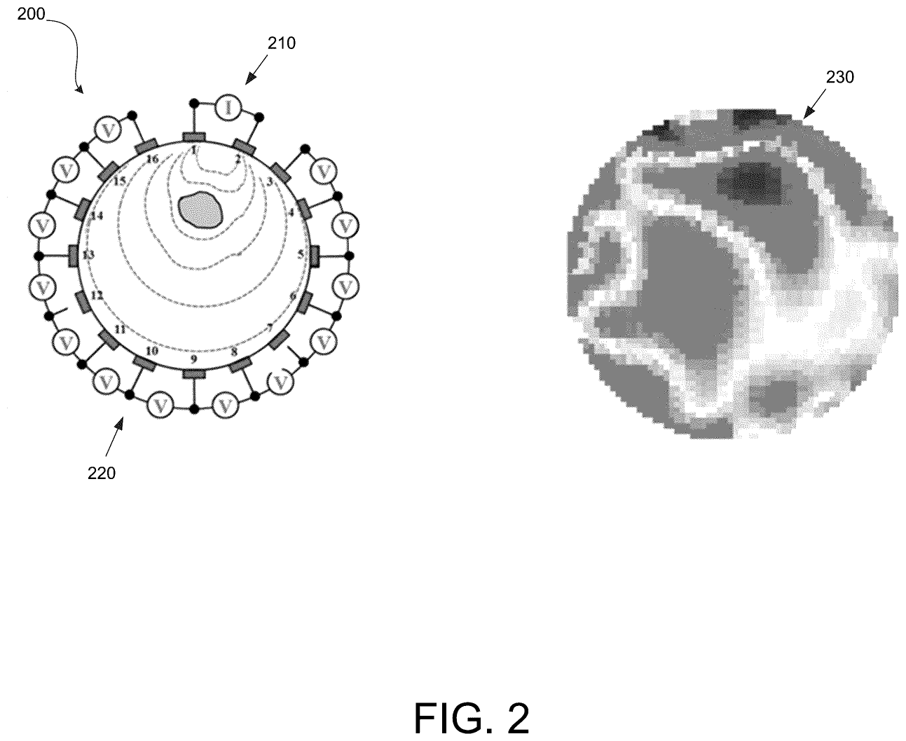

FIG. 2 is a diagram illustrating an example generation 200 of an electrical impedance tomograph. As shown in FIG. 2, the wrist is represented as a circle to which 16 electrodes are attached and are numbered 1, 2, . . . , 16. In some implementations, any number of electrodes other than 16 are used. At a first subset of electrodes 210, e.g., electrodes 1 and 2, an electrical current I is applied. The electrical current induces an electric field through the wrist, e.g., several cross-sections of the wrist, and this electric field provides an electric potential at each of the electrodes of a second subset of electrodes, e.g., electrodes 3-16. These electric potentials may be measured relative to a ground or a voltage between adjacent electrodes of the second subset may be measured (as pictured in FIG. 2).

Given the current and the electric potential at the electrodes (or the voltage between the electrodes), the electrical impedance through a cross-section (or several cross-sections) of the wrist may be determined using EIT. EIT is a noninvasive type of medical imaging in which the electrical conductivity, permittivity, and impedance of a part of the body is inferred from surface electrode measurements and used to form a tomographic image of that part. Electrical conductivity varies considerably among various biological tissues (absolute EIT) or the movement of fluids and gases within tissues (difference EIT). The majority of EIT systems apply small alternating currents at a single frequency.

The impedance as shown in FIG. 2 is characterized by a conductivity σ. This conductivity is a proportionality factor between an electric field (e.g., gradient of electric potential) and current density. As there are no sources of electric charge in the wrist, the gradient of the electric field is zero. In mathematical terms, the conductivity and electric potential satisfy the equation ∇·(σ∇ϕ)=0 inside the wrist, where ϕ is the electric potential. Solving this equation with an unknown conductivity σ directly is not possible. Nevertheless, one may find a conductivity σ by finding a value σ* through the wrist that satisfies (e.g., minimizes) the following functional.

$${‖ f ( Ω , σ ) - \tilde{ϕ} ‖}_{2}^{2} ,$$

where f is the restriction of potential ϕ to the boundary ∂Ω, over known σ and current source j, Ω is a wrist cross-section, and \tilde{ϕ} is a measured electric potential. The equation may be solved using finite element method (FEM) simulation. σ may then be inferred by performing the nonlinear optimization of the above functional. In some implementations, Ω represents a set of cross-sections and f is an over the cross-sections.

The result of minimizing the above functional for the conductivity is a map of the electric impedance 230 (which is simply σ⁻¹). The map is a two-dimensional image through the cross-section of the wrist. It is noted that because there are many material discontinuities inside the wrist at the various muscles, tendons, veins, bones, etc., the impedance will take on different values in each of the regions between those discontinuities.

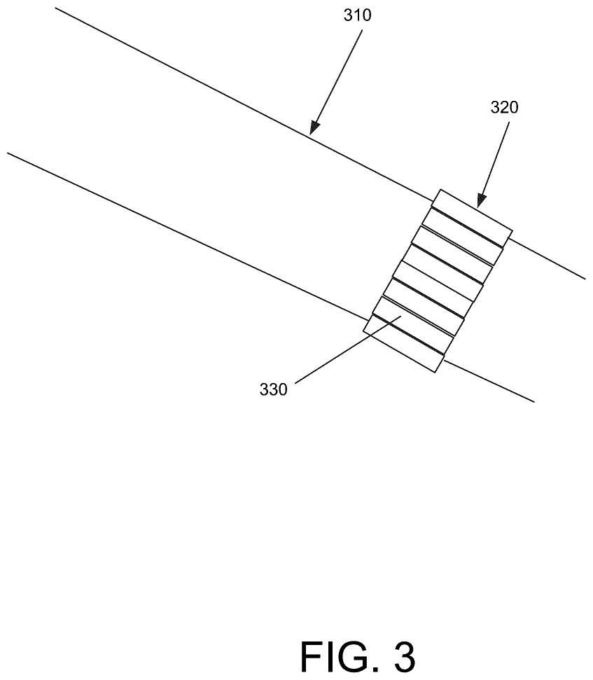

FIG. 3 is a diagram illustrating an example snug wristband 320 disposed around a wrist 310 with electrodes for determining an electrical impedance tomograph. While there are several possible housings for such electrodes, a snug (e.g., elastic) wristband such as those found on wristwatches provide a snug fit to wrists of any shape. In the example shown in FIG. 3, each electrode (e.g., electrode 330) is attached to the wristband such that the electrode is in good contact with the skin of the wrist, e.g., at least half of each electrode is in contact with the skin. It has been found that brass electrodes provided the best results for its high electrical conductivity and corrosion resistance at the same time (e.g. from sweat or oxidation over a long period of time), although other possibilities include copper, stainless steel, bronze, and titanium.

Again, the idea is that each electrical impedance tomograph maps to continuous hand kinematics at an instant of time. That is, a movement of the hand changes the impedance within the wrist. The continuous hand kinematics may be determined based on continually changing electrical impedance tomographs.

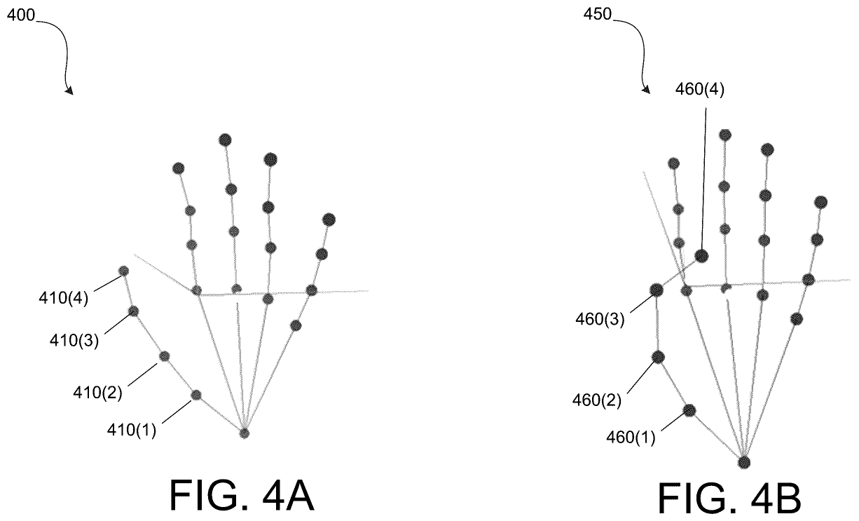

FIGS. 4A and 4B illustrate one way to represent the continuous hand kinematics. FIGS. 4A and 4B are diagrams illustrating example continuous hand kinematics using keypoints 400 and 450 in a human hand. For example, FIG. 4A shows 21 keypoints associated with locations on a human hand: four for each digit and a single keypoint on the base of the wrist. Of course, any number of keypoints may be used in this representation; 21 is for example purposes. A continuous hand kinematics may then be represented by the three-dimensional coordinates of each of the digit keypoints relative to the wrist keypoint. The continuous hand kinematics represented in FIG. 4A may be interpreted as an open hand, a “resting” gesture, with keypoints 410(1,2,3,4) being in a resting configuration.

In contrast, FIG. 4B shows the 21 keypoints arranged to represent a different gesture—a representation of the number “four” by moving the thumb in toward the middle finger. Now keypoints 460(1,2,3,4) have moved, with keypoint 460(4) in between the index and middle fingers. The coordinates of the 20 keypoints relative to the 21^{st} keypoint on the hand represents this and all other gestures for the purposes of the machine learning algorithm that takes an electrical impedance tomographs as input.

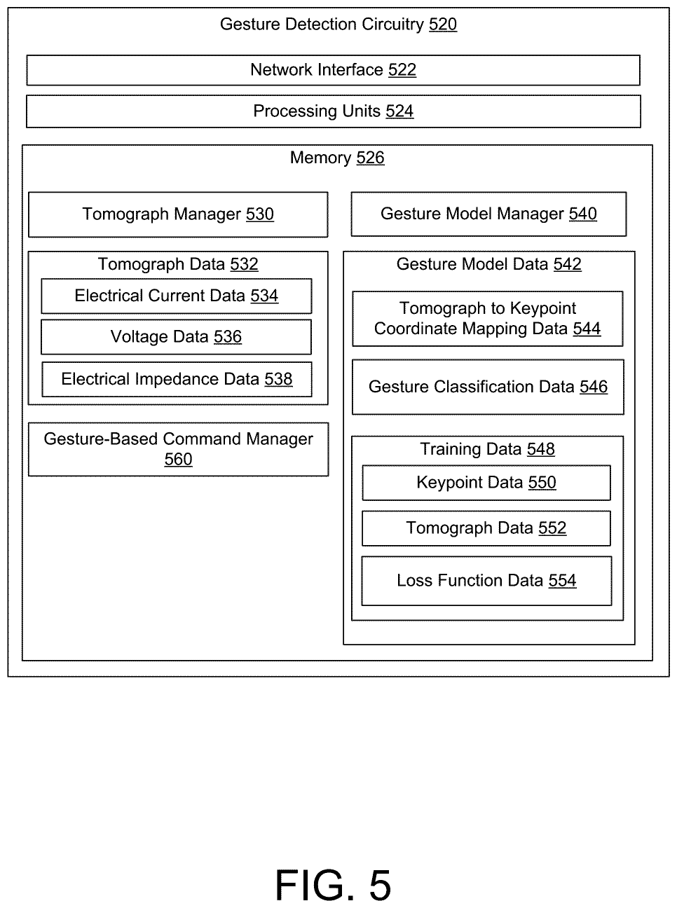

FIG. 5 is a diagram that illustrates example gesture detection circuitry 520 connected to smartglasses 100. The gesture detection circuitry 520 is configured to obtain (receive, generate) an electrical impedance tomograph and determine a gesture formed by the user while the tomograph was being generated.

The gesture detection circuitry 520 includes a network interface 522, one or more processing units 524, and nontransitory memory 526. The network interface 522 includes, for example, Ethernet adaptors, Bluetooth adaptors, and the like, for converting electronic and/or optical signals received from the network to electronic form for use by the gesture detection circuitry 520. The set of processing units 524 include one or more processing chips and/or assemblies. The memory 526 is a storage medium and includes both volatile memory (e.g., RAM) and non-volatile memory, such as one or more read only memories (ROMs), disk drives, solid state drives, and the like. The set of processing units 524 and the memory 526 together form processing circuitry, which is configured to carry out various methods and functions as described herein as a computer program product.

In some implementations, one or more of the components of the gesture detection circuitry 520 can be, or can include processors (e.g., processing units 524) configured to process instructions stored in the memory 526. Examples of such instructions as depicted in FIG. 5 include a tomograph manager 530, a gesture model manager 540, and a gesture-based command manager 560. Further, as illustrated in FIG. 5, the memory 526 is configured to store various data, which is described with respect to the respective managers that use such data.

The tomograph manager 530 is configured to obtain tomograph data 532 based on electrical current data 534 provided to a first subset of electrodes disposed on a wrist and voltage data 536 provided to a second subset of electrodes disposed on the wrist. In some implementations, the tomograph manager 530 is configured to receive the tomographs from an external source. In some implementations, the tomograph manager 530 is provided the current data 534 and voltage data 536 and generates the tomographs based on that data.

The gesture model manager 540 is configured to determine a continuous hand kinematics based on the electrical impedance data 538, e.g., an electrical impedance tomographs. The continuous hand kinematics is part of gesture model data 532 and is expressed as gesture classification data 546.

The gesture model is based on a convolutional neural network that has as an input layer an electrical impedance tomograph, e.g., electrical impedance data 538 and as an output layer a set of twenty keypoint coordinates representing a continuous hand kinematics. The electrical impedance data 538 is mapped to a set of twenty keypoint coordinates representing a continuous hand kinematics (e.g., tomograph to keypoint coordinate mapping data 544), and then the keypoint coordinates are classified as being a type of continuous hand kinematics. In an example model, there are six convolutional layers which use a leaky ReLU activation function and average pooling; other configurations, e.g., other numbers of layers, activation functions, etc., may be possible. In some implementations, the gesture model is based on a recurrent neural network. In some implementations, the gesture model is based on a graph neural network.

In some implementations, the gesture model may be trained by the gesture model manager 540 using training data 548. Training data 548 includes keypoint data 550 and corresponding tomograph data 552 as well as loss function data 554 that indicates a loss to be minimized when keypoint coordinates are determined for a given tomograph.

The gesture-based command manager 560 performs an action based on a continuous hand kinematics at a moment in time predicted by the gesture model manager 540. For example, a grasping motion may cause the gesture-based command manager 560 to search the AR display for an object within its field of view to grab, and then move the object through the field of view according to the continuous hand kinematics.

The components (e.g., modules, processing units 524) of gesture detection circuitry 520 can be configured to operate based on one or more platforms (e.g., one or more similar or different platforms) that can include one or more types of hardware, software, firmware, operating systems, runtime libraries, and/or so forth. In some implementations, the components of the gesture detection circuitry 520 can be configured to operate within a cluster of devices (e.g., a server farm). In such an implementation, the functionality and processing of the components of the gesture detection circuitry 520 can be distributed to several devices of the cluster of devices.

The components of the gesture detection circuitry 520 can be, or can include, any type of hardware and/or software configured to process attributes. In some implementations, one or more portions of the components shown in the components of the gesture detection circuitry 520 in FIG. 5 can be, or can include, a hardware-based module (e.g., a digital signal processor (DSP), a field programmable gate array (FPGA), a memory), a firmware module, and/or a software-based module (e.g., a module of computer code, a set of computer-readable instructions that can be executed at a computer). For example, in some implementations, one or more portions of the components of the gesture detection circuitry 520 can be, or can include, a software module configured for execution by at least one processor (not shown). In some implementations, the functionality of the components can be included in different modules and/or different components than those shown in FIG. 5, including combining functionality illustrated as two components into a single component.

Although not shown, in some implementations, the components of the gesture detection circuitry 520 (or portions thereof) can be configured to operate within, for example, a data center (e.g., a cloud computing environment), a computer system, one or more server/host devices, and/or so forth. In some implementations, the components of the gesture detection circuitry 520 (or portions thereof) can be configured to operate within a network. Thus, the components of the gesture detection circuitry 520 (or portions thereof) can be configured to function within various types of network environments that can include one or more devices and/or one or more server devices. For example, the network can be, or can include, a local area network (LAN), a wide area network (WAN), and/or so forth. The network can be, or can include, a wireless network and/or wireless network implemented using, for example, gateway devices, bridges, switches, and/or so forth. The network can include one or more segments and/or can have portions based on various protocols such as Internet Protocol (IP) and/or a proprietary protocol. The network can include at least a portion of the Internet.

In some implementations, one or more of the components of the search system can be, or can include, processors configured to process instructions stored in a memory. For example, tomograph manager 530 (and/or a portion thereof), gesture model manager 540 (and/or a portion thereof), and gesture-based command manager 560 (and/or a portion thereof) are examples of such instructions.

In some implementations, the memory 526 can be any type of memory such as a random-access memory, a disk drive memory, flash memory, and/or so forth. In some implementations, the memory 626 can be implemented as more than one memory component (e.g., more than one RAM component or disk drive memory) associated with the components of the gesture detection circuitry 620. In some implementations, the memory 526 can be a database memory. In some implementations, the memory 526 can be, or can include, a non-local memory. For example, the memory 526 can be, or can include, a memory shared by multiple devices (not shown). In some implementations, the memory 526 can be associated with a server device (not shown) within a network and configured to serve the components of the gesture detection circuitry 520. As illustrated in FIG. 5, the memory 526 is configured to store various data, including tomograph data 532 and gesture model data 542.

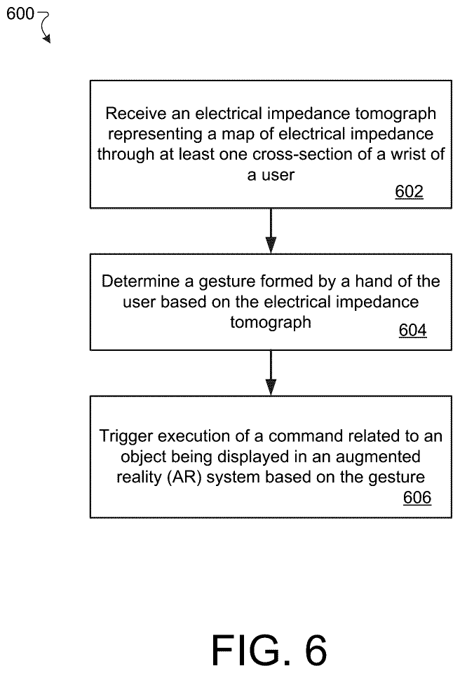

FIG. 6 is a flow chart illustrating a method 600 of determining a gesture based on an electrical impedance tomograph.

At 602, a tomograph manager (e.g., 530) receives an electrical impedance tomograph (e.g., tomograph data 538) representing a map of electrical impedance through at least one cross-section of a wrist of a user.

At 604. a gesture model manager (e.g., 540) determines a gesture formed by a hand of the user based on the electrical impedance tomograph.

At 606, a gesture-based command manager (e.g., 560) triggers execution of a command related to an object being displayed in an augmented reality (AR) system based on the gesture.

Specific structural and functional details disclosed herein are merely representative for purposes of describing example embodiments. Example embodiments, however, may be embodied in many alternate forms and should not be construed as limited to only the embodiments set forth herein.

The terminology used herein is for the purpose of describing particular embodiments only and is not intended to be limiting of the embodiments. As used herein, the singular forms “a,” “an,” and “the” are intended to include the plural forms as well, unless the context clearly indicates otherwise. It will be further understood that the terms “comprises,” “comprising,” “includes,” and/or “including,” when used in this specification, specify the presence of the stated features, steps, operations, elements, and/or components, but do not preclude the presence or addition of one or more other features, steps, operations, elements, components, and/or groups thereof.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present. As used herein the term “and/or” includes any and all combinations of one or more of the associated listed items.

Spatially relative terms, such as “beneath,” “below,” “lower,” “above,” “upper,” and the like, may be used herein for ease of description to describe one element or feature in relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 70 degrees or at other orientations) and the spatially relative descriptors used herein may be interpreted accordingly.

Example embodiments of the concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the described concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

Unless otherwise defined, the terms (including technical and scientific terms) used herein have the same meaning as commonly understood by one of ordinary skill in the art to which these concepts belong. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

While certain features of the described implementations have been illustrated as described herein, many modifications, substitutions, changes, and equivalents will now occur to those skilled in the art. It is, therefore, to be understood that the appended claims are intended to cover such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components, and/or features of the different implementations described.

What is claimed is:

1. A method, comprising:
receiving an electrical impedance tomograph representing a map of electrical impedance through at least one cross-section of a wrist of a user;
determining a gesture formed by a hand of the user based on the electrical impedance tomograph; and
triggering execution of a command related to an object being displayed in an augmented reality (AR) system based on the gesture.

2. The method as in claim 1, wherein determining the gesture includes:
inputting the electrical impedance tomograph into a machine learning classification engine, the machine learning classification engine being configured to map the electrical impedance tomograph to the gesture.

3. The method as in claim 2, wherein, in the machine learning classification engine, the hand is represented by a plurality of keypoints and the gesture is represented by an arrangement of the plurality of keypoints in space.

4. The method as in claim 1, wherein the electrical impedance tomograph is generated using a plurality of electrodes disposed on a band and in contact with the wrist of the user, the band being configured to fit snugly around the wrist of the user.

5. The method as in claim 4, where at least one of the plurality of electrodes includes brass.

6. The method as in claim 4, wherein receiving the electrical impedance tomograph includes:
applying an electrical current to a first subset of the plurality of electrodes;
determining electrical voltages between adjacent pairs of a second subset of the plurality of electrodes; and
generating the electrical impedance tomograph based on the electrical current at the first subset of the plurality of electrodes and the electrical voltages between adjacent pairs of the second subset of the plurality of electrodes.

7. The method as in claim 1, wherein the electrical impedance tomograph represents an average electrical impedance through a plurality of cross-sections of the wrist of the user.

8. An augmented reality (AR) system, including:
gesture detection circuitry coupled to a memory, the gesture detection circuitry being configured to:
receive an electrical impedance tomograph representing a map of electrical impedance through at least one cross-section of a wrist of a user; and
determine a gesture formed by a hand of the user based on the electrical impedance tomograph; and
wherein the AR system is configured to trigger execution of a command related to an object being displayed in the AR system based on the gesture.

9. The AR system as in claim 8, wherein the gesture detection circuitry configured to determine the gesture is further configured to:
input the electrical impedance tomograph into a machine learning classification engine, the machine learning classification engine being configured to map the electrical impedance tomograph to the gesture.

10. The AR system as in claim 9, wherein, in the machine learning classification engine, the hand is represented by a plurality of keypoints and the gesture is represented by an arrangement of the plurality of keypoints in space.

11. The AR system as in claim 8, wherein the electrical impedance tomograph is generated using a plurality of electrodes disposed on a band and in contact with the wrist of the user, the band being configured to fit snugly around the wrist of the user.

12. The AR system as in claim 11, where at least one of the plurality of electrodes includes brass.

13. The AR system as in claim 11, wherein receiving the electrical impedance tomograph includes:
applying an electrical current to a first subset of the plurality of electrodes;
determining electrical voltages between adjacent pairs of a second subset of the plurality of electrodes; and
generating the electrical impedance tomograph based on the electrical current at the first subset of the plurality of electrodes and the electrical voltages between adjacent pairs of the second subset of the plurality of electrodes.

14. The AR system as in claim 8, wherein the electrical impedance tomograph represents an average electrical impedance through a plurality of cross-sections of the wrist of the user.

15. A computer program product comprising a nontransitory storage medium, the computer program product including code that, when executed by processing circuitry, causes the processing circuitry to perform a method, the method comprising:
receiving an electrical impedance tomograph representing a map of electrical impedance through at least one cross-section of a wrist of a user;
determining a gesture formed by a hand of the user based on the electrical impedance tomograph; and
triggering execution of a command related to an object being displayed in an augmented reality (AR) system based on the gesture.

16. The computer program product as in claim 15, wherein determining the gesture includes:
inputting the electrical impedance tomograph into a machine learning classification engine, the machine learning classification engine being configured to map the electrical impedance tomograph to the gesture.

17. The computer program product as in claim 16, wherein, in the machine learning classification engine, the hand is represented by a plurality of keypoints and the gesture is represented by an arrangement of the plurality of keypoints in space.

18. The computer program product as in claim 15, wherein the electrical impedance tomograph is generated using a plurality of electrodes disposed on a band and in contact with the wrist of the user, the band being configured to fit snugly around the wrist of the user.

19. The computer program product as in claim 18, wherein receiving the electrical impedance tomograph includes:
applying an electrical current to a first subset of the plurality of electrodes;
determining electrical voltages between adjacent pairs of a second subset of the plurality of electrodes; and
generating the electrical impedance tomograph based on the electrical current at the first subset of the plurality of electrodes and the electrical voltages between adjacent pairs of the second subset of the plurality of electrodes.

20. The computer program product as in claim 15, wherein the electrical impedance tomograph represents an average electrical impedance through a plurality of cross-sections of the wrist of the user.
